(19) United States
(12) Patent Application Publication
(10) Pub. No.: US 2015/0027448 A1
(43) Pub. Date: Jan. 29, 2015

(54) APPLICATOR DEVICE FOR APPLYING A URISHEATH TO A PENIS

(71) Applicant: Securin ApS, Hillerød (DK)

(72) Inventor: Brian Nielsen, Gorlose (DK)

(21) Appl. No.: 14/360,868

(22) PCT Filed: Nov. 23, 2012
(86) PCT No.: PCT/EP2012/073500

§ 371 Date: May 27, 2014

Related U.S. Application Data
(60) Provisional application No. 61/563,716, filed on Nov. 25, 2011.

(30) Foreign Application Priority Data
Nov. 25, 2011 (DK) PA 2011 70654
Sep. 12, 2012 (DK) PA 2012 70564

(51) Int. Cl. A61F 6/04; A61F 6/00

(52) U.S. Cl. CPC A61F 6/04; A61F 6/005; A61F 2006/049
USPC 128/844

(57) ABSTRACT

An applicator device (20) suitable for applying a urisheath (21) to a penis, said applicator device comprising: a first flange (41), a second flange (42), and a third flange (43), a first displaceable element (31) connected to said first flange, a second displaceable element (32) connected to said second flange and a third displaceable element (33) connected to said third flange. Said displaceable elements being arranged such that said first, second and third flanges are displaceable with respect to each other, and a urisheath (21) is mountable thereon, and that said first, second and third flanges are displaceable between a first position where the urisheath has a first perimeter and a second position where the urisheath has a second perimeter which is at least 20% larger than the first perimeter. Said applicator further comprises a displacing mechanism (51,52,61,62,63,64,65,66,67,68) which is arranged such that said first, second and third flanges are displaceable relative to each other, in a manner such that the first flange is displaceable along a first vector direction, the second flange is displaceable along a second vector direction which has a non-zero vector component perpendicular to the first vector direction and the third flange is displaceable along a third vector direction C which has a non-zero vector component parallel to the first vector direction but opposite in direction, and/or a non-zero vector component which is perpendicular to the first vector direction, but opposite the direction of the non-zero vector component of the second vector direction.

The current invention relates to an applicator device suitable for applying a urisheath to a penis, said applicator device comprising: a first flange, a second flange, and a third flange, a first displaceable element connected to said first flange, a second displaceable element connected to said second flange and a third displaceable element connected to said third flange, said displaceable elements being arranged such that said first, second and third flanges are displaceable with respect to each other, and such that said first, second and third flanges are arranged such that a urisheath is mountable around said first, second and third flanges and that said first, second and third displaceable elements are arranged such that the first, second and third flanges are displaceable between a first position where the urisheath has a first perimeter and a second position where the urisheath has a second perimeter which is larger than the first perimeter.

### DESCRIPTION OF RELATED ART

Urisheaths are commonly of the type which are applied to the penis in a manner similar to a condom. They are applied to the tip of the penis with a rolled up portion and then they are rolled backwards towards the base of the penis. Applicators for these types of urisheaths are known in the prior art. For example FR2752158, U.S. Pat. No. 5,713,880, WO8802624 and EP0569287.

However, recently other forms of urisheaths have been developed. For an example, please see the inventor's co-pending application WO 2011/098581. These new urisheaths are designed to be placed at the base of the penis and then unrolled in a direction away from the base of the penis and towards the tip of the penis. In order to create a good seal on the penis the urisheath needs to have a diameter which is smaller than the penis diameter. In order to apply the urisheath at the base of the penis, the urisheath needs to be stretched in its rolled up form so that it can be placed in its rolled up form at the base of the penis. In order to make this as easy as possible, it is necessary to stretch the urisheath to a diameter which is larger than the penis diameter and then hold this enlarged diameter while placing the urisheath on the penis. This has created a demand for a new type of applicator device for allowing a stretching operation which is greater than that known in the art.

### SUMMARY OF THE INVENTION

It is therefore a first aspect of the current invention to provide a new type of applicator device which is improved with respect to the prior art. This is solved with an applicator device as mentioned in the opening paragraph which further comprises the features of the characterizing portion of claim 1. In this way, an applicator is provided which can provide a greater degree of stretch than prior art applicators with a smaller amount of motion of the individual flanges. Also, a more circular stretch is possible, instead of a rectangular stretch as is the case with prior art solutions. For a discussion of how the vectors mentioned in the claims would appear, please refer to the discussion with regards to FIGS. 1 to 4 below.

A more precise definition of the relationship between the displacement vectors of the flanges could be that the angle between any two adjacent displacement vectors should be less than 180 degrees. This is true both for embodiments with three flanges and for embodiments with four flanges. In fact, this relationship would be relevant for any number of flanges. The definition could also be that the angle between adjacent displacement vectors of the flanges should be less than 150 degrees, or less than 140 degrees, or less than 130 degrees or less than or equal to 120 degrees.

In one embodiment, the second perimeter could be at least 20% larger than said first perimeter. In other embodiments, the second perimeter could be at least 30% larger, at least 40% larger or at least 50% larger than the first perimeter.

In one embodiment, the applicator could be arranged such that the minimum diameter between the inner surfaces of the flanges in the second position of the displaceable elements is at least 30 mm. In other embodiments the minimum diameter is at least 35 mm, 37 mm, 40 mm, 42 mm or 45 mm. It should be noted that the minimum diameter between the inner surfaces of the flanges should be interpreted as the diameter of the cylindrical object having the largest diameter which can be inserted between the flanges in the second position without contacting any parts of the inner surfaces of the flanges.

In order to make the application of the urisheath to the penis easier, the applicator device could comprise a holding mechanism which is arranged to hold said first, second and third flanges in said second position. In this way, the user could put the applicator device from its first position to the second position by applying a force, and then release the force since the holding mechanism will keep the flanges in the second position.

It should be noted that according to this specification a “displacing mechanism” should be understood as a mechanism which translates a force into a displacement of the displaceable elements. For example, in the embodiment shown in FIG. 5, a tension force is applied between the two finger holes 51,52. This force is translated into relative motion of the displaceable elements. Within the scope of this application, the displacing mechanism could take many forms. Two non-limiting examples are a) a screw mechanism where a user can apply torque to a screw mechanism which then forces the displaceable elements apart and b) a squeeze mechanism where a user can apply a squeezing force to a squeeze mechanism (for example a ring) which then forces the displaceable elements apart.

It can also be noted that for the sake of this specification, in cases where the displacement of the displaceable elements occurs along a nonlinear path, for example a curved path, then the displacement vector for the sake of the claims should be the vector formed between the position of the displaceable element in the first position and the position of the displaceable element in the second position. This could be called the “effective displacement vector of the flanges”. The position of the displaceable element to be used for determining the first and second position could be defined by the centre of the flange attached to the displaceable element.

It should be emphasized that the term “comprises/comprising/comprised of” when used in this specification is taken to specify the presence of stated features, integers, steps or components but does not preclude the presence or addition of one or more other features, integers, steps, components or groups thereof. For example, in the claims it is stated that the applicator comprises three flanges, however this should be understood as at least three flanges, since applicators having 3, 4, 5, 6, and and so on flanges should be included in the scope of protection.

### BRIEF DESCRIPTION OF THE DRAWINGS

In the following, the invention will be described in greater detail with reference to embodiments shown by the enclosed figures. It should be emphasized that the embodiments shown are used for example purposes only and should not be used to limit the scope of the invention.

FIGS. 1 and 2 schematically show one conceptual embodiment of an applicator according to the invention in order to explain the terms of the claims.

FIGS. 3 and 4 schematically show another conceptual embodiment of an applicator according to the invention in order to explain the terms of the claims.

FIG. 5 shows a front top perspective view of a first embodiment of an applicator according to the invention in a stretched position.

FIG. 6 shows a front view of the applicator shown in FIG. 5.

FIG. 7 shows a side view of the applicator shown in FIG. 5.

FIG. 8 shows a rear view of the applicator shown in FIG. 5.

FIG. 9 shows a rear top perspective view of the applicator shown in FIG. 5.

FIG. 10 shows a front exploded view the applicator shown in FIG. 5.

FIG. 11 shows a front perspective exploded view the applicator shown in FIG. 5.

FIG. 12 shows a rear exploded view the applicator shown in FIG. 5.

FIG. 13 shows a rear perspective exploded view the applicator shown in FIG. 5.

FIG. 14 shows a front view the applicator shown in FIG. 5, but in a retracted position.

FIG. 15 shows a front perspective view of the applicator shown in FIG. 14.

FIG. 16 shows a rear view of the applicator shown in FIG. 14.

FIG. 17 shows a rear perspective view of the applicator shown in FIG. 14.

FIG. 18 shows a schematic exploded front view of a second embodiment of an applicator according to the invention.

FIG. 19 shows a schematic front view of a third embodiment of an applicator according to the invention in a retracted position.

FIG. 20 shows a schematic front view of the applicator of FIG. 19 but in a stretched position.

FIG. 21 shows a schematic front view of a fourth embodiment of an applicator according to the invention in a retracted position.

FIG. 22 shows the applicator of FIG. 21 in an expanded position.

FIGS. 23a-23d show an embodiment of a method for using an applicator according to the current invention together with a traditional urisheath.

FIGS. 24a-24d show another embodiment of a method for using an applicator according to the current invention with a traditional urisheath.

### DETAILED DESCRIPTION OF THE EMBODIMENTS

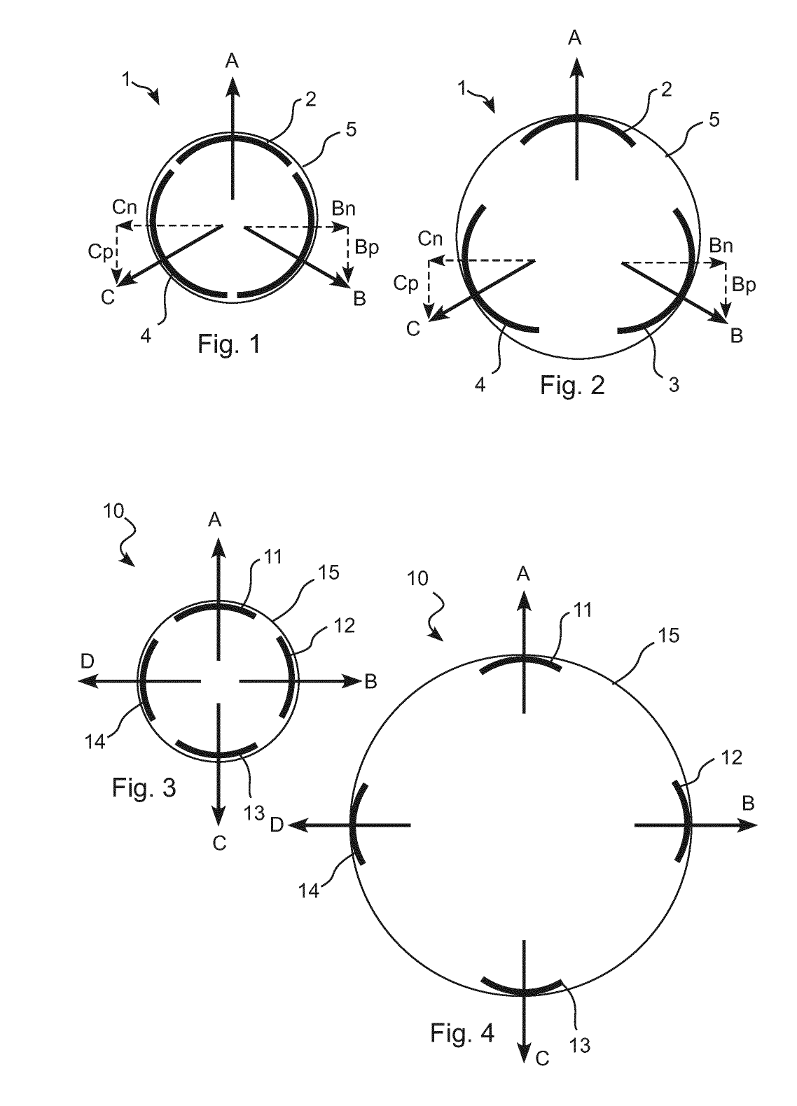

FIGS. 1 through 4 are provided to clarify the terms used in the claims. FIGS. 1 and 2 show a schematic embodiment of an applicator 1 with three flanges 2,3,4. A urisheath 5 is shown arranged around the flanges 2,3,4. As can be seen in the figures, when the flanges are displaced outwardly to stretch the urisheath, the first flange 2 moves along a vector A, the second flange 3 moves along a vector B and the third flange 4 moves along a vector C. The vector B comprises a vector component Bn which is non-zero and perpendicular (normal) to the vector A. The vector B also comprises a vector component Bp which is parallel to the vector A, but opposite in direction to the vector A. The vector C comprises a vector component Cp which is non-zero and parallel to the vector A, but opposite in direction to the vector A. The vector C also comprises a vector component Cn which is perpendicular (normal) to the vector A. In addition, it can be seen that the angles between adjacent displacement vectors is always less than 180 degrees. In this example where the vectors are equally spaced, the angle between adjacent displacement vectors is 120 degrees for all adjacent vectors.

FIGS. 3 and 4 show a schematic embodiment of an applicator 10 with four flanges, 11,12,13,14 and a urisheath 15 arranged around the flanges. As can be seen in the figures, when stretching the urisheath the first flange 11 moves along a vector A, the second flange 12 moves along a vector B, the third flange 13 moves along a vector C and the fourth flange 14 moves along a vector D. In this embodiment, the vector B is arranged completely perpendicular to the vector A, the vector C is arranged completely parallel to the vector A, but opposite in direction to the direction of vector A and the vector D is arranged perpendicular to the vector A (or parallel to the vector B), but opposite in direction to the direction of vector B. In this example the displacement of each flange is orthogonal to the other flanges. It can also be said that the angles between adjacent displacement vectors in this embodiment are all 90 degrees.

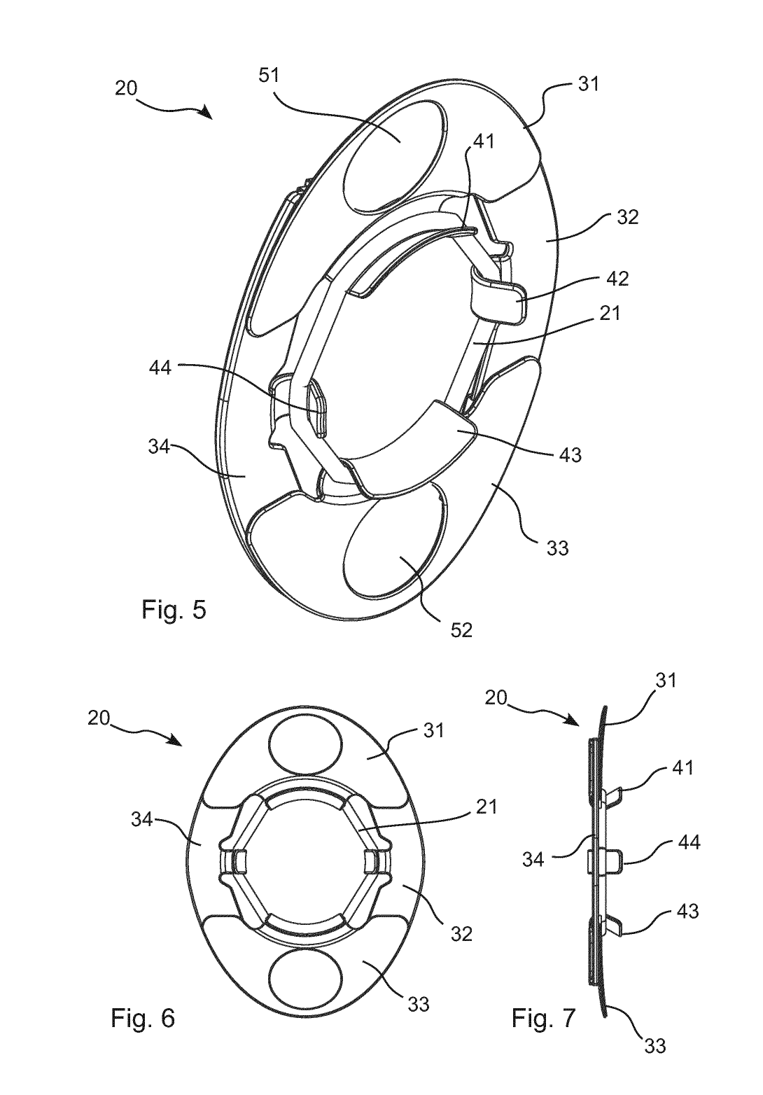

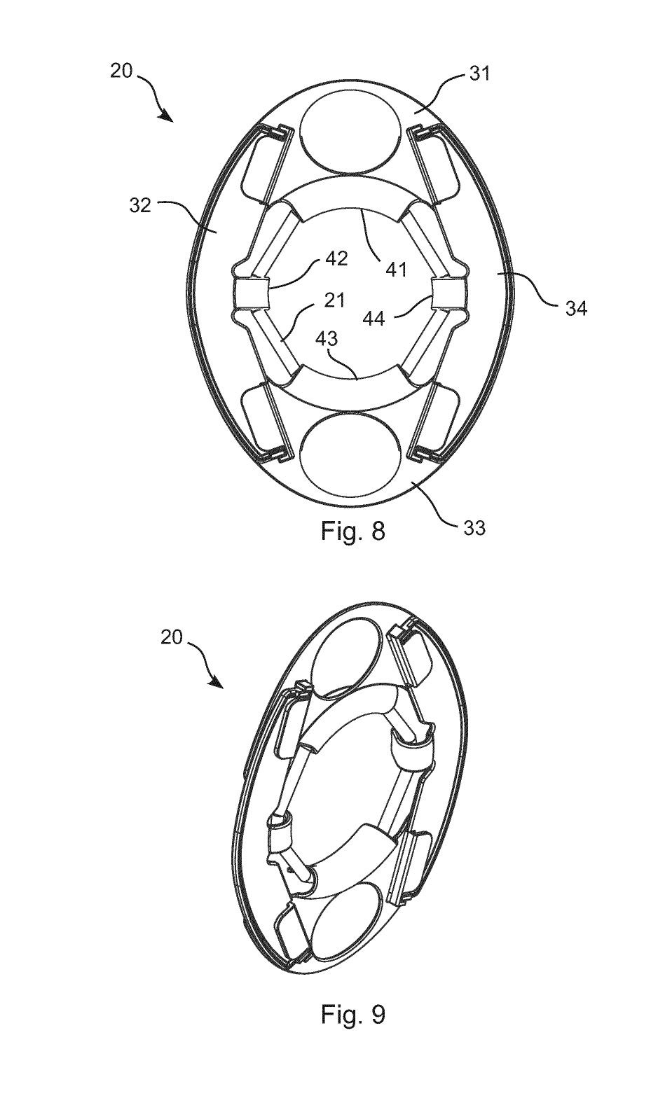

FIGS. 5-17 show different views of an embodiment of an applicator 20 according to the invention in different positions.

FIGS. 5-9 show the applicator 20 in an expanded or stretched position where the urisheath 21 is stretched. The urisheath 21 is shown as a ring since in practise, the typical urisheath used with this embodiment will be formed from a cylindrical tube made from a flexible material and where the cylindrical tube is rolled up to form a ring, similar to a condom.

The applicator 20 comprises four displaceable elements 31,32,33,34. Each displaceable element is provided with a flange. According to the wording of the claims, the applicator therefore comprises a first flange 41 on the first displaceable element 31, a second flange 42 on the second displaceable element 32, a third flange 43 on the third displaceable element 33 and a fourth flange 44 on the fourth displaceable element 34. The flanges in this embodiment are formed as hooks around which the urisheath can be mounted. When the displaceable elements are displaced away from each other, the flanges pull the urisheath and cause it to stretch.

In the current embodiment, the flanges all have a length which is adjacent the perimeter of the urisheath. The longer the length of the flange, the more support the urisheath has on the flange. In the current embodiment, the first and third flange 41,43 are longer than the second and fourth flanges 42,44.

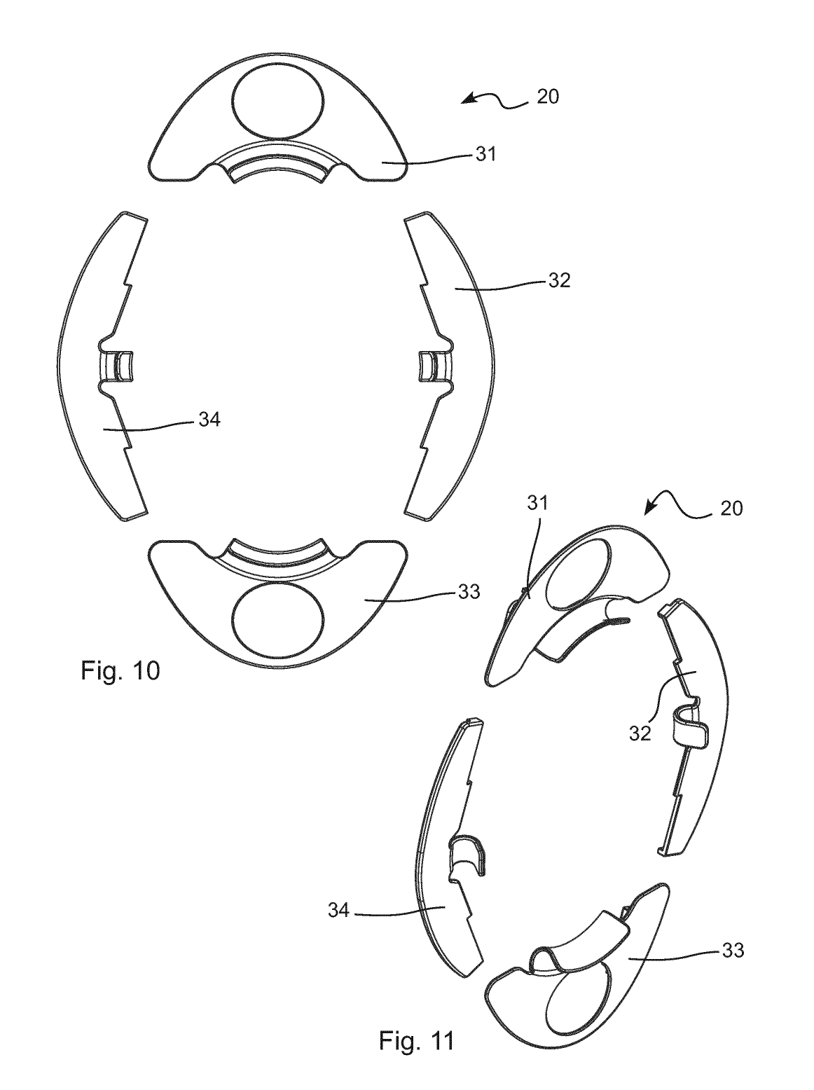

FIGS. 10-13 show different exploded views of the applicator 20 showing the four displaceable elements 31,32,33,34 in more detail.

FIGS. 14-17 show different views of the applicator 20 in a relaxed state or in a non-stretched state. A urisheath is not shown in the figures, but could be imagined as being arranged around the four flanges 41,42,43,44. As can be seen by comparing for example FIG. 15 and FIG. 5, when the applicator is moved from its relaxed state (FIG. 15) to its expanded state (FIG. 5) the flanges move apart from each other in order to stretch the urisheath 21.

The motion of the flanges 41,42,43,44 are similar to the motions as discussed with regards to the schematic embodiment shown in FIGS. 3 and 4. The vector directions A,B,C,D have been marked on FIG. 14.

Due to the design of the applicator of FIGS. 5-17, the user of the applicator can apply a force which pulls the first and third displaceable elements 31,33 apart from each other which then forces the second and fourth displaceable element 32,34 apart from each other. In order to apply the force in an easy way, finger holes 51,52 are provided in the first and third displaceable elements.

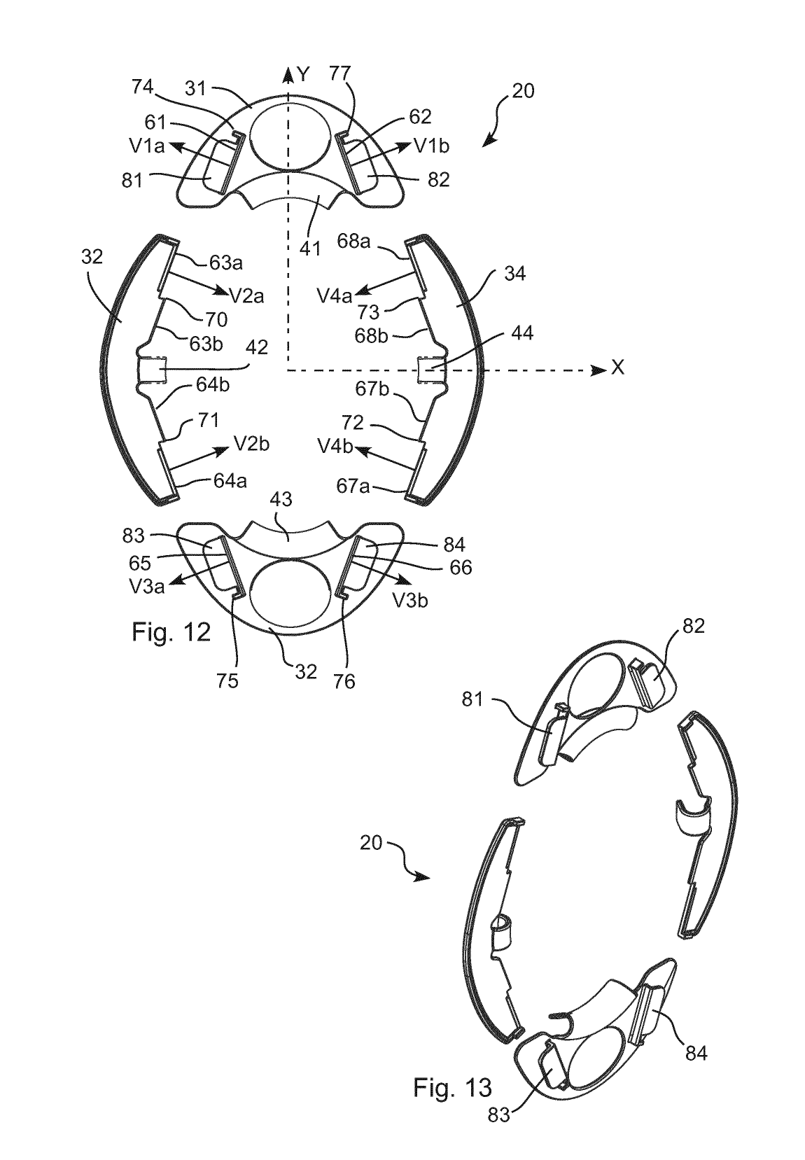

The design of the applicator which ensures the motion of the second and fourth displaceable elements 32,34 can best be explained with respect to FIGS. 12 and 13. As shown in these figures, the motion is provided due to the interaction between sloped surfaces on the displaceable elements. The first displaceable element 31 is provided with a sloped surface 61 to the left of the centre line which has a normal vector V1a having a positive y and a negative x component and a sloped surface 62 to the right of the centreline Y having a normal vector V1b having a positive y component and a positive x component. The second displaceable element 32 has a first surface 63 above the centre line X having a normal vector V2a having a positive x and a negative y component and a second surface 64 below the centre line X having a normal vector V2b having a positive y component and a positive x component. The third displaceable element 33 has a first surface 65 to the left of the centre line Y with a normal vector V3a having a negative x component and a negative y component and a second surface 66 to the right of the centre line Y having a normal vector V3b having a positive x and a negative y component. The fourth displaceable element 34 comprises a first surface 67 below the centre line X having a normal vector V4a having a positive y and a negative x component and a second surface 68 above the centre line X having a normal vector V4b having a negative x and a negative y component.

When the applicator is assembled, the vectors V1a and V2a are parallel, the vectors V2b and V3a are parallel, the vectors V3b and V4b are parallel and the vectors V4a and V1b are parallel.

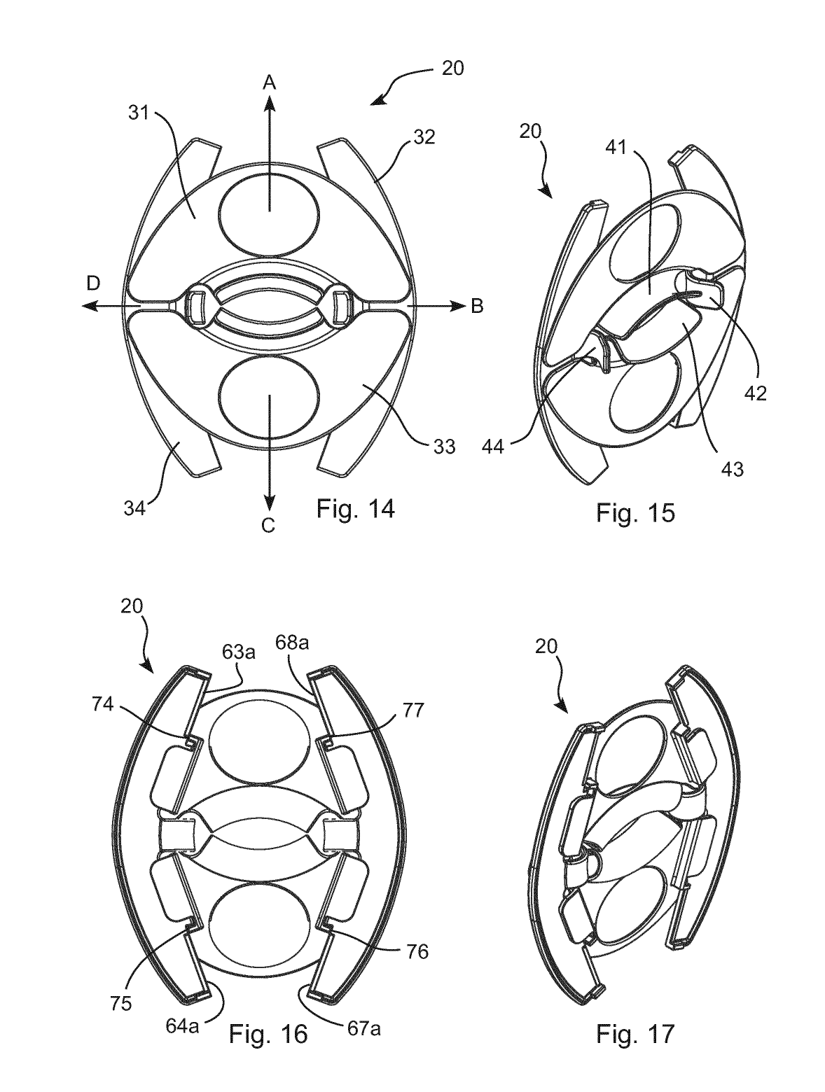

As can be seen from FIG. 12 for example, in this embodiment of the applicator the sloped surfaces of the second and fourth displaceable elements are formed with a step 70, 71, 72, 73 such that the surface is divided into a first portion 63a, 64a, 67a, 68a at a first level and a second portion 63b, 64b, 67b, 68b at a second level. Furthermore the sloped surfaces 61,62,65,66 of the first and third displaceable elements 31,33 are provided with a protrusion 74,75,76,77 at the end of the sloped surface 61,62,65,66. In the rest position (see for example FIG. 16), the protrusions 74,75,76,77 of the first and third displaceable element are slideably arranged on the first portion 63a, 64a, 67a, 68a of the sloped surfaces of the second and fourth displaceable elements and the sloped surfaces 61,62,65,66 of the first and third displaceable elements are slideably arranged on the second portions 63a, 64a, 67a, 68a of the sloped surfaces of the second and fourth displaceable elements.

As the first and third displaceable elements are pulled apart, the sloped surfaces slide along each other, thereby pushing the second and fourth displaceable elements apart, thereby further stretching the urisheath.

When the first and third displaceable elements are pulled a certain distance, the protrusions 74,75,76,77 move past the end of the first portion 63a,64a, 67a,68a of the sloped surfaces of the second and fourth displaceable elements thereby locking the displaceable elements such that they cannot retract to the rest position again. Furthermore, the end of the sloped surfaces of the first and third displaceable elements also move past the step 71,72,73,74 of the sloped surfaces of the second and fourth displaceable elements with provides a further lock. It can however be mentioned that by pulling the second and fourth displaceable elements apart, the locks release and the applicator can again be put into its rest state. In order to hold the device together, the sloped surfaces 61,62,65,66 are provided with flanges 81,82,83,84 which protrude over the sloped surfaces 63,64, 67,68 of the second and fourth displaceable elements.

It can also be mentioned that the urisheath itself, ensures that the displaceable elements are held together and do not fall apart. As soon as the urisheath is removed, the displaceable elements fall apart and can be removed from the penis. This is due to the fact that the displaceable elements in the current embodiment are arranged as individual elements which are detachable from each other when the urisheath is removed from the flanges. In other embodiments it could be imagined that some of the displaceable elements were more securely fastened together and that at least one displaceable element could be removed such that the applicator could be removed from the penis after application. In other embodiments, the displaceable elements could remain connected to each other after the urisheath was applied, by lifting the applicator off the penis. In another embodiment, the applicator could be formed so that it did not entirely encircle the penis.

It should also be noted that the current embodiment, as can be seen in for example FIG. 7, is arranged such that it is slightly curved. In other words, the displaceable elements are arranged such that at least in the extended position the applicator has a form which is curved when seen from the side. The curved form makes it easier to place the applicator at the very base of the penis right up close to the body.

Furthermore, it should also be noted that the applicator in this embodiment is formed as a form of shield element which protects the pubic hair from the patient from getting tangled with the urisheath during the unrolling process.

In another embodiment (not shown), a thin elastic film is applied to the rear side (body facing side) of the applicator with a hole in it. The hole is centred with respect to the applicator. When the applicator is put into its extended position, the thin elastic film is stretched and the hole is also stretched. The penis is then inserted in the hole. In this way, the thin elastic film provides a totally closed shield element thereby totally preventing pubic hair from contacting the urisheath.

The purpose of the shield element is firstly to prevent pubic hairs from getting entangled in the urisheath, but the shield will also prevent unnecessary contact between the person applying the urisheath and the person receiving the urisheath. The shield element can be defined as an element which is integrated into the applicator in a way which at least partially covers the annular area of the body of the user of the urisheath which extends from the base of the penis and outwards. In one definition one could say that the shield element is arranged to at least partially cover an annular area of the body of the user of the urisheath with an inner diameter equal to the outer diameter of the penis rod and a width of at least 0.5 cm, at least 1 cm or at least 2 cm. In one embodiment “at least partially covers” could be interpreted as covering at least 30% of the area, at least 50% of the area, at least 75% of the area or at least 90% of the area. In one embodiment, at least 50% of the circumference of the penis is covered by the shield element.

It can also be noted that the shield element can be integrated in the applicator in different ways. In one embodiment, the shield element is split into multiple parts, each part integrated with one of the displaceable elements. This is the form taken by the embodiment shown in FIGS. 5-17. In this embodiment, the displaceable elements are formed as plate like elements which provide the shield element function. In another embodiment, the shield element could be only attached to one or more of the displaceable elements. In an embodiment where the shield element is only attached to one element, then the shield element should be formed as a plate element with an opening, the centre of the opening being arranged essentially co-axially with the centre of the urisheath when the applicator is in its extended position. In the embodiment shown in FIGS. 5-15, gaps are formed between the flanges and the plate like portions of the displaceable elements. However, in other embodiments, the plate like elements could extend under the flanges to cover more of the annular area around the penis. In one embodiment, the innermost (closest to the penis during application) edges of the shield element could be formed from a thin flexible material which allows the shield element to adapt to the penis and thereby cover the annular area around the penis completely.

The locking functionality as described above could be described as a “one step” ratchet mechanism, where a ratchet mechanism according to the current specification is understood as a mechanism which allows motion in one direction while preventing motion in the reverse direction. In another embodiment (not shown) multiple stops could be imagined, for example the sloped surfaces of the second and fourth displaceable elements could be divided into more portions with more steps thereby providing a ratchet mechanism having more locking steps.

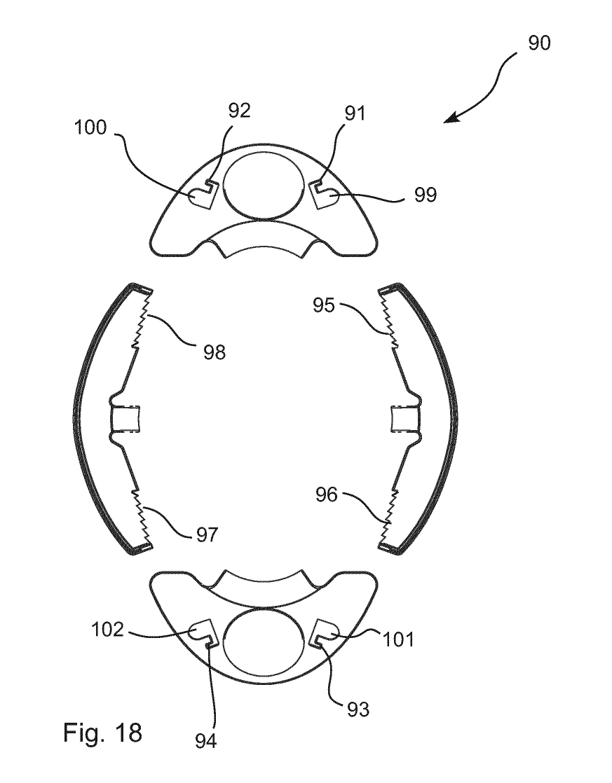

Furthermore, in the current embodiment, sloped surfaces are provided on both the first and third displaceable elements and the second and fourth displaceable elements. However, in another embodiment sloped surfaces could be provided on only the first and third displaceable elements or only on the second and fourth displaceable elements. On the element without a sloped surface, a simple protrusion could be arranged which slides on the corresponding sloped surface. The embodiment 90 shown in FIG. 18 is a simple schematic example of such an embodiment. Two protrusions 91,92,93,94 are provided on the first and third elements and two sloped surfaces 95,96,97,98 with a number of ratchet steps are provided on the second and fourth displaceable elements. A simple flange 99,100,101,102 is further provided on the first and third displaceable elements to prevent the displaceable elements from falling apart.

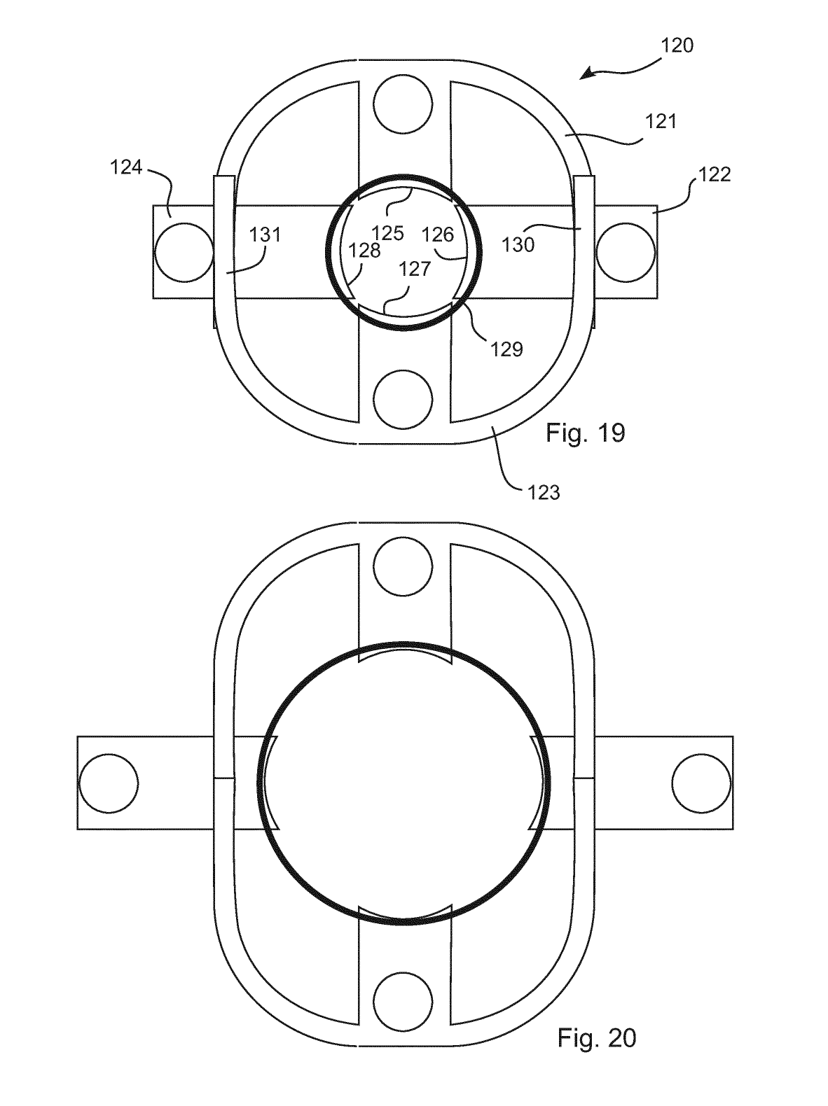

The embodiment of FIGS. 19 and 20 show a very simple and schematic embodiment 120 of an applicator according to the current invention. In this embodiment, the applicator again comprises a first displaceable element 121, a second displaceable element 122, a third displaceable element 123 and a fourth displaceable element 124. A first flange 125 is connected to the first displaceable element 121, a second flange 126 is connected to the second displaceable element 122, a third flange 127 is connected to the third displaceable element 123 and a fourth flange 128 is connected to the fourth displaceable element 124. A urisheath 129 is mounted around the flanges.

When the applicator is to be used the user first pulls the first and third displaceable elements apart after which the user pulls the second and fourth displaceable elements apart. Ratchet mechanisms 130,131 between the first and third displaceable elements ensure that the first and third displaceable elements maintain their position once they have been pulled apart. An additional ratchet mechanism between the second displaceable element and the first displaceable element and an additional ratchet mechanism between the fourth displaceable element and the first displaceable element ensure that the second and fourth displaceable elements maintain their position once they have been pulled apart. The main difference between this embodiment and the previous embodiment is that the user has to first pull in one direction and then in another direction, thereby providing a two-step procedure whereas in the previous embodiment, the expansion procedure was a single step procedure.

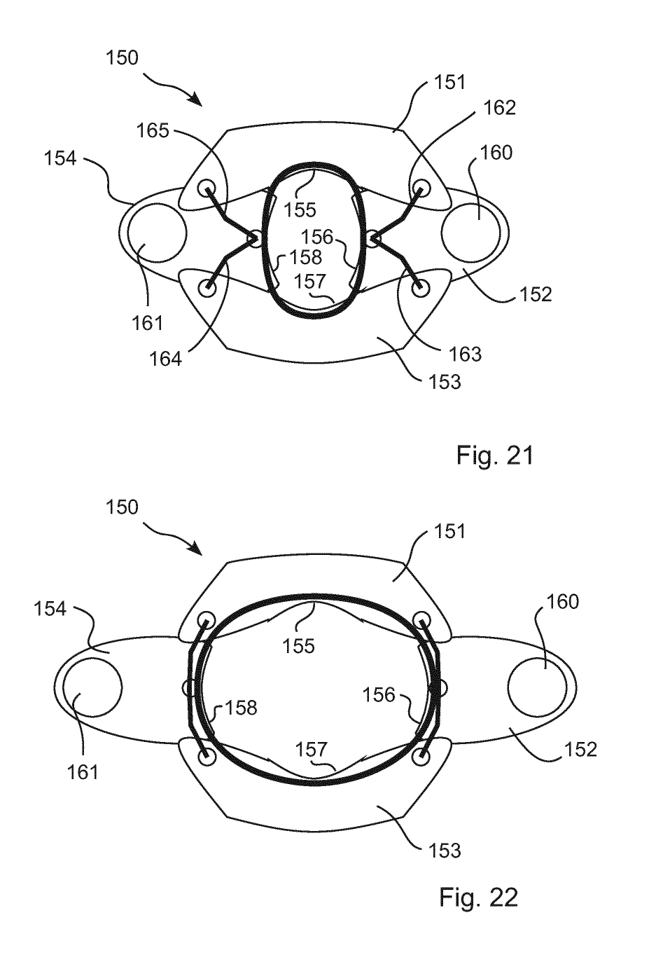

The embodiment of an applicator 150 schematically shown in FIGS. 21 and 22 shows another mechanism possibility for providing an applicator where the user only has to apply a force in one direction, but where the applicator stretches the urisheath in more than one direction. In this embodiment, linkages are used.

The applicator 150 of this embodiment comprises a first displaceable element 151, a second displaceable element 152, a third displaceable element 153 and a fourth displaceable element 154. A first flange 155 is connected to the first displaceable element 151, a second flange 156 is connected to the second displaceable element 152, a third flange 157 is connected to the third displaceable element 153 and a fourth flange 158 is connected to the fourth displaceable element 154. A urisheath 159 is mounted around the flanges.

The displaceable elements are connected together via linkage mechanisms. When the second and fourth displaceable elements 152,154 are pulled apart via the finger holes 160,161, the third and fourth displaceable elements are pushed apart in a direction perpendicular to the motion of the second and fourth displaceable elements, as shown by comparing FIGS. 21 and 22.

This motion is provided by providing four links in a linkage mechanism. A first link 162 is pivotably fastened at a first end to the first displaceable element and pivotably connected at a second end to the second displaceable element. A second link 163 is pivotably fastened at a first end to the second displaceable element and pivotably connected at a second end to the third displaceable element. A third link 164 is pivotably fastened at a first end to the third displaceable element and pivotably fastened at a second end to the fourth displaceable element. A fourth link 165 is pivotably fastened at a first end to the fourth displaceable element and pivotably fastened at a second end to the first displaceable element. In the rest position the links are arranged at a 90 degree angle to each other in this embodiment, whereas in the extended position, they are pivoted via the motion of the first and third displaceable elements such that they are parallel. In order to achieve a greater motion of the second and fourth displaceable elements, the angle of the links could start at a smaller angle.

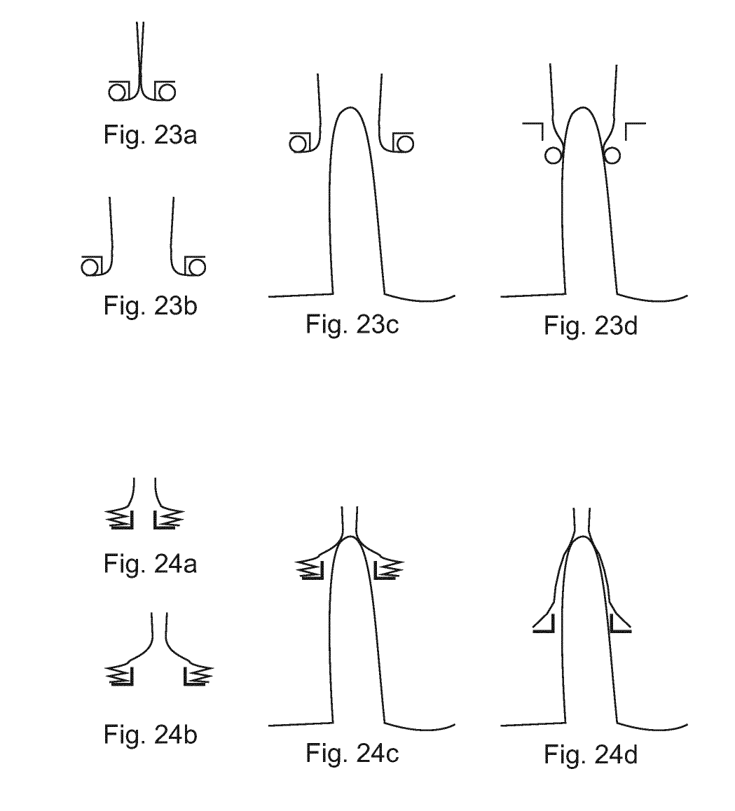

The current embodiments have all described applicators together with the new type of urisheaths which are applied at the base of the penis and then unrolled towards the tip. However, an applicator according to the current invention could also be used with traditional urisheaths. In one embodiment (FIG. 23a-23d), a traditional urisheath is mounted on the applicator (FIG. 23a), the applicator expanded (FIG. 23b), then the urisheath placed on the head of the penis (FIG. 23c). The rolled up portion of the urisheath is then pushed off the flanges of the applicator (FIG. 23d), the applicator removed and then the rolled up portion unrolled. In another embodiment (FIGS. 24a-24d), instead of having a rolled up urisheath on the applicator, a urisheath which is folded could be mounted on the applicator (FIG. 24a). The applicator is then expanded (FIG. 24b). The applicator is then placed over the penis (FIG. 24c) and the applicator pushed down over the penis (FIG. 24d) as the urisheath unfolds from the applicator.

It is to be noted that the figures and the above description have shown the example embodiments in a simple and schematic manner. Specific details have not been shown since the person skilled in the art should be familiar with these details and they would just unnecessarily complicate this description.

It should also be noted that this specification has shown a number of independent features which could form the basis of divisional applications. For example, in the current claim set, the feature which forms the base of the claims is the third flange. However, an applicator with a holding mechanism could form the basis of one divisional application. In another example, an applicator with a displacing mechanism similar to the one shown in FIGS. 5-17 or a displacing mechanism similar to the one shown in FIGS. 21-22 could be imagined having only two flanges (second and fourth with respect to the mechanism of FIGS. 5-17 or first and third with respect to the mechanism of FIGS. 21-22) could also form the basis of a divisional application.

Finally it can also be noted that the embodiments in this specification have all been related to applicators for urisheaths, however it should be noted that the applicator as claimed in the claims could also be used as an applicator for condoms. We note that since the applicator displaces in more directions than prior art condom applicators, a condom would be stretched more uniformly with the current applicator and a user would be required to displace the displacement mechanism less than in prior art condom applicators to achieve a similar enlargement. Furthermore, if the applicator is combined with a shield element as described in this application, then the condom applicator would also provide a way of minimizing contact between the person applying the condom and the person receiving the condom. This could be especially relevant in certain situations, such as the adult film industry and in the sex industry.

When using the applicator as a condom applicator, the amount of stretch required is less than with urisheaths. This would allow a smaller version of the applicator to be produced than the one shown in the figures of this application. In this respect, it could also be possible to form the applicator such that the displacement mechanism could be operated with one hand. This could for example be provided by replacing the two holes 51 with finger hooks on one side of the applicator. The user could then grasp the finger hooks with one of his or her fingers and his or her thumb and then stretch the applicator. This type of embodiment would be especially relevant for situations where no holding mechanism is provided, however, the current embodiment with two finger holes would also be relevant in certain cases, especially in the case where a holding mechanism is provided. It should be noted that should the applicator be used for applying condoms, it could be advantageous to form the flanges differently than in the manner shown in the figures. However, flanges for condom adapters are well known in the art. One example is provided in EP1933784.

1. An applicator device suitable for applying a urisheath to a penis, said applicator device comprising:
a. a first flange, a second flange, and a third flange,
b. a first displaceable element connected to said first flange, a second displaceable element connected to said second flange and a third displaceable element connected to said third flange,
c. said displaceable elements being arranged such that said first, second and third flanges are displaceable with respect to each other, and
d. such that said first, second and third flanges are arranged such that a urisheath is mountable around said first, second and third flanges and that said first, second and third displaceable elements are arranged such that the first, second and third flanges are displaceable between a first position where the urisheath has a first perimeter and a second position where the urisheath has a second perimeter which is at least 20% larger than the first perimeter,
characterized in that
e. said applicator further comprises a displacing mechanism which is arranged such that said first, second and third flanges are displaceable relative to each other, in a manner such that
f. the first flange is displaceable along a first vector direction A,
g. the second flange is displaceable along a second vector direction B which has a non-zero vector component Bn which is perpendicular to the first vector direction A
h. the third flange is displaceable along a third vector direction C which has a non-zero vector component Cp which is parallel to the first vector direction A but opposite in direction to the first vector direction A and/or a non-zero vector component Cn which is perpendicular to the first vector direction A, but opposite the direction of the non-zero vector component Bn of the second vector direction B.

2. An applicator device according to claim 1, characterized in that said applicator further comprises a holding mechanism which is arranged to hold said first, second and third flanges in said second position.

3. An applicator device according to claim 1, characterized in that said applicator device further comprises a fourth flange and a fourth displaceable element connected to the fourth flange where the displacing mechanism is arranged such that the fourth flange is displaceable relative to the first, second and third flanges along a fourth vector direction D which has a non-zero vector component which is perpendicular to the first vector direction A and opposite in direction to the non-zero vector component Bn of the second vector direction B.

4. An applicator device according to claim 3, characterized in that the second vector direction B is perpendicular to the first vector direction, the third vector direction C is parallel but opposite in direction to the first vector direction A and the fourth vector direction D is parallel but opposite in direction to the second vector direction B.

5. An applicator device according to claim 2, characterized in that the holding mechanism is a form of ratchet mechanism having at least one ratchet stop.

6. An applicator device according to claim 1, characterized in that the displacing mechanism comprises a ramp element having a sloped surface on one of said first, second, third displaceable elements which interacts with a sliding element on another one of said first, second or third displaceable elements, such that motion of one displaceable element along a first direction (A,C) causes motion of another displaceable element along a direction (C,D) which has a non-zero vector component which is perpendicular to the first direction.

7. An applicator device according to claim 1, characterized in that the applicator also comprises a shield element.

8. An applicator device according to claim 1, characterized in that the angle between any two adjacent displacement vectors (A,B,C,D) is less than 180 degrees.

9. A kit of parts comprising an applicator device according to claim 1 and a urisheath mounted around the first, second and third flanges.

10. A kit of parts according to claim 9 characterized in that the urisheath is of the kind comprising a tube made from a thin and elastic material which is rolled up and mounted around the first, second and third flanges such that the end of the urisheath which will be located furthest from the base of the penis after unrolling is inside the rolled up portion and that the end of the urisheath which will be closest to the base of the penis after unrolling is on the outside of the rolled up portion.

11. A kit of parts comprising an applicator device according to claim 1 and a condom mounted around the first, second and third flanges.
